(19) United States
(12) Patent Application Publication
(10) Pub. No.: US 2023/0265523 A1
(43) Pub. Date: Aug. 24, 2023

(54) MOLECULAR MARKER FOR EARLY PANCREATIC NEOPLASM DETECTION, DETECTION METHOD AND USE THEREOF

(71) Applicant: KANTE (SHENZHEN) BIOTECHNOLOGIES, ING, Shenzhen, Guangdong (CN)

(72) Inventors: Xiaoying XIAO, Dalian (CN); Guishan XIAO, Dalian (CN)

(21) Appl. No.: 17/997,568

(22) PCT Filed: Apr. 9, 2021
(86) PCT No.: PCT/CN2021/086188

§ 371 Date: Oct. 31, 2022

(30) Foreign Application Priority Data
Apr. 29, 2020 (CN) 202010356498.0

(51) Int. Cl. C12Q 1/6886; C12Q 1/686

(52) U.S. Cl. CPC C12Q 1/6886; C12Q 1/686; C12Q 2600/158; C12Q 2600/178

(57) ABSTRACT

This description relates to a molecular marker for early pancreatic neoplasm detection, a detection method and use thereof. The pancreatic cancer marker miRNAs: miR-30c, miR-24, miR-23a and miR-132. The combination, method and kit provided by the present invention can be used for screening and differential diagnosis of early pancreatic cancer, monitoring of disease complications and recurrence, evaluation of curative effect, drug efficacy and guidance of precise drug use, etc., and it has the advantages of a wide detection spectrum, high sensitivity, good specificity, low detection cost, convenient material acquisition, and easy storage of samples. This method can be widely used in the early screening and prognosis of pancreatic cancer, improving the low specificity and low sensitivity caused by the individual differences that are difficult to overcome due to the instability of a single marker or biomarkers currently widely used in clinical practice.

### RELATED APPLICATIONS

The present application is a U.S. National Phase of International Application Number PCT/CN2021/086188, filed Apr. 9, 2021, and claims priority to Chinese Application Number 202010356498.0, filed Apr. 29, 2020.

### INCORPORATION BY REFERENCE

The sequence listing provided in the file entitled NSequence_2022102002_v2.txt, which is an ASCII text file that was created on Oct. 20, 2022, and which comprises 3,529 bytes, is hereby incorporated by reference in its entirety.

### TECHNICAL FIELD

The invention belongs to the field of biotechnology and clinical molecular diagnostic drug development, and particularly relates to methods and kits capable of distinguishing early lesions of pancreatic cancer from pancreatitis and intraductal papillary mucinous neoplasm lesions of the pancreas and normal human blood, PCR (polymerase chain reaction) or any other detection method using these markers.

### BACKGROUND

Early pancreatic cancer usually refers to pancreatic cancer with a tumor diameter [symbol] 2.0 cm, no lymph node metastasis, no pancreatic capsule and peripancreatic infiltration, and no invasion of blood vessels and adjacent organs. The stage is T1aN0M0. However, some scholars believe that most pancreatic cancers with a size of 1.0 cm to 2.0 cm have had lymph node metastasis, and advocate that the tumor diameter [symbol] <1.0 cm is the standard for early pancreatic cancer. Unless the lesion happens to be located at the duodenal papilla, symptoms of bile and pancreatic duct obstruction can appear early, and there are few clinical symptoms. Other scholars have proposed that the definitions of early pancreatic cancer and small pancreatic cancer are different, the latter mainly refers to the maximum diameter of the tumor 2.0 cm, regardless of whether there is lymph node metastasis. Therefore, the diagnosis of early pancreatic cancer should focus on the screening of high-risk groups, molecular biological diagnosis and the exploration of new imaging methods.

At present, the most widely used clinical diagnostic method for pancreatic cancer is to use a variety of imaging methods to identify tumors in patients with suspected pancreatic cancer, including abdominal B-ultrasound, computed tomography (CT), magnetic resonance imaging (MRI), and endoscopic ultrasound (EUS) and Positron Emission Tomography (PET). Due to the fact that pancreatic cancer tumors are located deep in the human body, there is no obvious clinical manifestation in the early stage, and it is difficult to diagnose with radiographic imaging in the stage of small cancer foci. As a result, most pancreatic cancer patients have entered the advanced stage when they are diagnosed, and the lesions have already metastasized. The patient loses the opportunity for surgical treatment.

Considering the limitations of imaging detection methods, researchers began to think about whether some biomolecules in the human body could be used as diagnostic targets to improve the specificity of diagnosis. Therefore, in recent years, people are keen to develop new biomarkers. There are also many studies on biomarkers for pancreatic cancer, but few have been proven to be effective in detecting early-stage pancreatic cancer. Currently, carbohydrate antigen 19-9 (CA 19-9) is the most widely used biomarker for pancreatic cancer. However, since CA19-9, CA125, CA50, TIMP-1, and CEA have low diagnostic sensitivity and specificity for pancreatic cancer (all around 30-40% and 60%), they are not the best diagnostic marker for screening and early detection of pancreatic cancer, and its main clinical application is as a marker for monitoring disease progression and treatment response.

Although CN101942502A and the inventor's prior application CN109423519A in the prior art, disclose the use of microRNAs for detecting the diagnosis of early clinical pancreatic cancer, in the prior art, a single detection index is used to determine, and the single miRNAs invented is not the most sensitive, and it is not the most reflective of the actual situation of clinical patients, the lack of comprehensive consideration of the individualized clinical characterization of each miRNA may lead to the fact that its product test results cannot truly reflect the actual situation of the patient, resulting in missed diagnosis and misdiagnosis.

Traditional miRNA detection techniques are mainly Northern Blotting, microarray, in situ hybridization (ISH) and nucleic acid amplification techniques. Today, although many advances have been made in the development of miRNA detection technology, the following problems still need to be solved:

1) At present, although many miRNA detection methods have been reported, the technology that can be commercialized and extended to the clinic is mainly PCR. However, ordinary RT-PCR cannot achieve the high sensitivity, accuracy and specificity required in practice, and in the face of large-scale clinical samples, how to achieve rapid and large-scale detection is a technical difficulty;

2) Usually a miRNA can regulate multiple functions at the same time and is closely related to multiple diseases. Therefore, it is very important to develop high-throughput detection technology that can detect multiple target miRNAs simultaneously;

3) At present, blood is often used as a test sample for in vitro miRNA analysis in clinical practice. This generally requires the extraction and isolation of miRNA from serum, but the content of miRNA is low, and RNA degradation is prone to occur, so it is necessary to further develop an RNA extraction technology that can provide high-quality miRNA samples.

Therefore, development of efficient and sensitive miRNA and detection methods is of great significance for the early diagnosis, treatment and prognosis of pancreatic cancer.

### SUMMARY OF THE INVENTION

In view of the difficulties of early diagnosis of pancreatic cancer and the low sensitivity and specificity of existing biomarkers, the present invention provides a new method for screening and detecting pancreatic cancer by differential expression of miRNAs in a group of serum, and a new use of early pancreatic neoplasm molecule markers.

The inventive concept of the present invention is: miRNA is a nucleotide of 18-24 nucleotides in length composed of non-protein-encoding small RNAs, and involves regulating the expression or inhibition of multiple genes by degrading target regulatory mRNAs or polypeptides, thereby regulating various tumors processes, including cell proliferation, migration, invasion, survival and metastasis. The abnormal expression of miRNA is reflected in various stages in the pathogenesis of pancreatic cancer, and the expression level at the molecular level is different. Therefore, according to the differential expression of miRNAs of different pathological types, detecting characteristic diseases at various stages is a technical method with excellent sensitivity and specificity to distinguish patients suffering from pancreatic benign diseases or pancreatic cancer.

In order to achieve the above objects, the miRNAs of pancreatic cancer markers claimed in the present invention are: miR-30c, miR-24, miR-23a and miR-132.

The miR-30c comprises hsa-miR-30c-5p; miR-24 comprises hsa-miR-24-3p; miR-23a comprises hsa-miR-23a-3p; miR-132 comprises hsa-miR-132-3p.

The above miRNAs primers and probe sequences are:

| SEQ ID | | | |
|---|---|---|---|
| NO. | miRNAs | | SEQUENCE |
| SEQ ID | miR-30c | Probe | TCAGCCCTATCACCGTTGTC |
| NO. 1 | | | |
| | | Forward | CGGTTGTAAACATCCTACACT |
| | | Reverse | GGGTCCGAGGTATCCAT |
| | | RT | CCTATCACCGTAAGCAGGGTC |
| | | primer | CGAGGTATCCATCGCACGCGT |
| | | | GGACAACGGTGATAGGATCTGC |
| SEQ ID | miR-24 | Probe | TCAACAGCTCCTATGGACGAC |
| NO. 2 | | | C |
| | | Forward | CTTGGCTCAGTTCAGCAG |
| | | Reverse | GAGGTATCCATCGCACG |
| | | RT | CTCCTATGGACAAGCAGGGTCC |
| | | primer | GAGGTATCCATCGCACGCGAGG |
| | | | GTCGTCCATAGGAGCTGTTCCT |
| SEQ ID | miR-23a | Probe | TCCCCAGCATAACC |
| NO. 3 | | | CTCG |
| | | Forward | TATCACATTGCCAGGGAT |
| | | Reverse | GGGTCCGAGGTATCCAT |
| | | RT | CCAGCATAACCAAGCAGGGTC |
| | | primer | CGAGGTATCCATCGCACGCAT |
| | | | CCGAGGGTTATGCTGGGGAAA |
| | | | T |
| SEQ ID | miR-132 | Probe | TTCCGCCTGCTTGACTGTAGC |
| NO. 4 | | | CGACG |
| | | Forward | TTCCTAACAGTCTACAGCCAT |
| | | Reverse | GGGTCCGAGGTATCCAT |
| | | RT | CCTGCTTGACTAAGCAGGGTCC |
| | | primer | GAGGTATCCATCGCACGTCGGC |
| | | | TACAGTCAAGCAGGCGACCATG |

Another object of the present invention is to claim a core diagnostic combination comprising any one or a combination of 2 to 4 of the above-mentioned miRNAs respectively.

The combination comprising:

Combination 1: miR-24/miR-23a/miR-132/miR-30c;

Combination 2: miR-130/miR-200c/miR-154/miR-30c;

Combination 3: miR-24/miR-132/miR-1207/let-7i;

Combination 4: miR-30c/miR-24/miR-59/miR-132;

Combination 5: miR-130/miR-21/let-7i/miR-30c;

Combination 6: miR-30c/miR-154/miR-23a/miR-57.

The invention not only finds out the best combination for early clinical diagnosis of pancreatic cancer, but also finds out the best clinical classification treatment method for pancreatic cancer patients.

The present invention claims to protect the use of the above-mentioned early-stage pancreatic cancer diagnostic molecular markers, that is, the above-mentioned early-stage pancreatic cancer markers in a kit or in any other convenient method, comprising but not limited to a portable test strip, a digital test strip/card and a detector, as well as a use of any chemical method to modify derivatives of the above-mentioned miRNA molecule. The kit or other detection methods comprises any one of miRNAs probe combinations for detecting early-stage pancreatic cancer markers as mentioned above.

In the present invention, the multi-combination joint detection results are combined with the conventional pathological results to comprehensively determine the detection accuracy, and the detection accuracy is high (>95%). However, in the prior art, only a single detection index (a single microRNA is used to judge), and the miRNAs in the prior art are not the most sensitive, and the lack of comprehensive consideration of individualized clinical characteristics may lead to product test results that cannot truly reflect the actual condition of the patient.

Another object of the present invention is to claim the early screening and precise drug detection methods using the above-mentioned miRNAs. On the basis of screening out a group of miRNAs that are abnormally highly expressed in pancreatic cancer patients, they are used as candidate markers for early detection of pancreatic cancer. In the detection process, considering the cost and convenience of operation, the present invention adopts the widely used TaqMan probe method real-time fluorescent quantitative PCR technology. Compared with the SYBR Green I dye method, the TaqMan probe method has advantages in specificity and sensitivity. Given the low content of miRNAs in human serum, improving the specificity of detection is a major challenge. In order to improve the detection specificity and avoid false positive results, the inventor independently designed the specific probes and primers for target miRNAs, according to the principle of Taqman probe technology; secondly, in order to reduce the error caused by the separate detection of internal reference and target in ordinary Taqman probe detection, the present invention adopts multiple probe technology to react the internal reference (U6) and target miRNA in the same system, which not only greatly reduces the operation error, but also avoids the inconvenience caused by the scarcity of clinical samples to a certain extent. Since there is no mature reagent system for detecting miRNA in serum with multiple Taqman probes on the market, we have spent a lot of time and energy to optimize the existing RNA extraction, reverse transcription and PCR reagent systems on the market. Types are repeatedly tested to establish a stable standard operational procedure (SOP).

The specific steps of the miRNAs early screening and precise drug detection system construction method of the present invention are as follows:

S1. Screen pancreatic cancer miRNA, design the specific probe and primer for the target miRNA according to the principle of TaqMan probe technology; use U6 or hsa-miR-16 or hsa-miR-159a as the internal reference to react with the target miRNA in the same system;

S2. Optimization of miRNA extraction technology

S2.1 Determine the extraction reagent, according to the quality of miRNA extracted from the test sample by the separation kit, to select the best extraction reagent as TRIzol LS Reagent;

S2.2 Optimize the influencing factor in the extraction process, the main factors affecting the quality of miRNA extraction are: the amount of lysate, the amount of chloroform, the amount of isopropanol, and the centrifugation conditions, etc, the TRIzol LS Reagent kit was used to investigate effects of different isopropanol dosages and centrifugation conditions on the quality of extracted miRNA, and designing three detailed optimization schemes;

S2.3 optimizes the details of the conventional extraction method of TRIzol LS according to the above three schemes;

According to the amount of isopropanol, centrifugation time and dosage, three optimization schemes and comparison schemes are designed. The difference between scheme A and the traditional scheme is that the centrifugation time is 20 min and the centrifugation is 20000 g; the difference between scheme B and the traditional scheme is that the dosage of isopropanol is 200 μL, 600 μL, and 800 μL; the difference between Scheme C and the traditional scheme is that the dosage of isopropanol is 800 μL, the centrifugation time is 20 min, and the centrifugation is 20000 g.

S3. Optimization and establishment of multiplex RT-qPCR system reaction procedure and reaction system

S3.1 RNA loading optimization for reverse transcription

To make gradient settings for the target miRNA loading amount, and determine that the optimal loading amount of reverse-transcribed RNA is 50 ng;

S3.2 Optimization of PCR Amplification Reagents

AceQ qPCR Probe Master Mix and Premix Ex Taq™ were used for the comparison experiments using normal human serum and pancreatic cancer patient serum respectively, and Premix Ex Taq™ was selected as the qPCR reagent for clinical sample detection.

The present invention also claims to protect a method for clinical diagnosis using the detection system constructed by the above method, the specific steps are:

Collect clinical samples and enroll eligible cases

S2. RNA extraction

(1) Add 600 ul TRIzol™ LS to each 200 ul serum sample and incubate at room temperature to fully lyse;

(2) Add chloroform to the lysate and incubate at room temperature; centrifuge at 20,000 g, 4° C. for 20 min, and transfer the upper aqueous phase to a new centrifuge tube;

(3) Add 800 μL of isopropanol, incubate at room temperature; centrifuge at 12,000×g for 10 min at 4° C., RNA forms a white precipitate at the bottom of the tube, remove the supernatant; add 75% ethanol to resuspend and wash the precipitate; 7500×g, centrifuge at 4° C. for 5 min, remove the supernatant and air dry; add ddH₂O to dissolve RNA; determine the concentration and quality of the extracted RNA.

S3. RT-PCR reaction procedure and reaction system

(1) Prepare the following system in a 0.1 ml 8-strip PCR tube, pipetting and mixing, and prepare multiple samples together and then dispense:

| Reagent | Amount (μl) |
|---|---|
| RNA | X (50 ng) |
| 10x Buffer | 1.5 |
| dNTP mix | 0.15 |
| RT enzyme | 1 |
| RNase inhibitor | 0.19 |
| U6 RT primer (5 μM) | 1 |
| miRNA RT primer (5 μM) | 1 |
| RNase-free ddH₂O | 10.16-X |
| Total Volume | 15 |

Any miR comprising miR-30c, miR-24, miR-23a, and miR-132, is combined with U6, according to the ratio in the table, the working solution is prepared firstly, and then the corresponding reaction reagent is added according to the ratio in the table to ensure the total volume was 15 microliters; then, the PCR amplification experiment was performed; the following procedure was performed in the PCR amplification instrument: 16° C. 30 min→42° C. 30 min→85° C. 5 min →4° C., and centrifuged slightly to the bottom of the tube after completion.

(2) Prepare the following system in a 0.2 ml PCR tube or RNAase-free 1.5 ml EP tube and mix by pipetting

| Reagent | Amount (μl) |
|---|---|
| cDNA | 3 |
| Premix Ex Taq (Probe qPCR) (2×) | 5 |
| U6 Forward Primer (10 μM) | 0.2 |
| U6 Reverse Primer (10 μM) | 0.2 |
| miRNA Forward Primer (10 μM) | 0.2 |
| miRNA Reverse Primer (10 μM) | 0.2 |
| U6 Probe (10 μM) | 0.4 |
| miRNA Probe (10 μM) | 0.4 |
| RNase-free ddH2O | 0.4 |
| Total Volume | 10 |

A two-step method was used for PCR amplification, the reaction conditions are: pre-denaturation, 1 cycle, 95° C. for 30 seconds, PCR reaction, 40 cycles, 95° C. for 5 seconds, 60° C. for 30 seconds, annealing at 50° C. for 30 seconds, 1 cycle.

(3) QuantStudio DX real-time fluorescence quantitative PCR system, the reaction conditions are: pre-denaturation, 1 cycle, 95° C. for 30 seconds, PCR reaction, 45 cycles, 95° C. for 5 seconds and 60° C. for 40 seconds.

| Reagent | Amount (μl) |
|---|---|
| cDNA | 3 |
| Premix Ex Taq (Probe qPCR) (2×) | 5 |
| ROX Reference Dye (50×) | 0.2 |
| U6 Forward Primer (10 μM) | 0.2 |
| U6 Reverse Primer (10 μM) | 0.2 |
| miRNA Forward Primer (10 μM) | 0.2 |
| miRNA Reverse Primer (10 μM) | 0.2 |
| U6 Probe (10 μM) | 0.4 |
| miRNA Probe (10 μM) | 0.4 |
| RNase-free ddH2O | 0.2 |
| Total Volume | 10 |

S4. According to the quantitative PCR results performed with the core diagnostic combination, analyze the distribution of biomarkers in the patient's blood sample, and determine the patient's pathological status.

Compared with the prior art, the beneficial effects of the present invention are:

(1) The miRNAs of the present invention are the most effective biomarker combinations found through systematic research, repeatedly using different samples, and verified by multiple research centers and clinical centers, miR-30c, miR-24, miR-23a and miR-132 are proposed as the diagnostic biomarkers of pancreatic cancer, which provided theoretical support for early pancreatic neoplasm molecular diagnosis and precision medicine.

(2) In view of miRNAs with the characteristics of short sequence, low tissue content, high homology, etc., correspondingly higher requirements for detection technology are put forward. The present invention establishes a Taqman probe multiplex real-time fluorescence quantitative PCR system to overcome the above problem, so as to achieve the purpose of convenient, rapid and highly specific detection of multiple target miRNAs in serum samples, to reliably distinguish early pancreatic cancer from pancreatitis lesions and normal people, and to provide technical support for the early detection of pancreatic cancer and new ideas for the development of miRNA markers.

(3) The present invention establishes a multi-combination joint detection, that is, 6 combination joint detection results are combined with conventional pathological results to comprehensively judge the detection accuracy, and the detection accuracy is high (>98%). The detection method is based on the best method for clinical diagnosis and precise treatment based on the verification of nearly 600 cases in 5 major clinical research centers in China.

The present invention is a biological small molecule developed based on the difference in expression intensity of various miRNAs in early pancreatic cancer cells, which can be used for molecular diagnosis and treatment of early pancreatic cancer. The invention provides a variety of early pancreatic cancer marker combinations and early screening and precise drug detection methods. The pancreatic cancer marker provided by the present invention includes the combined expression intensity of four microRNAs that are stably present and detectable in the serum/plasma and saliva of the subject.

The technical solution provided by the present invention is different from any miRNAs and screening solutions in the prior art, through clinical multi-center verification, inventor integrates patient medical background, BMI (obesity index), living habits (drinking and smoking), clinical metabolic index hemogram and its variability in individual and follow-up individual hemograms, using big data to comprehensively analyze the contribution of each miRNAs to the canceration of early pancreatic cells, and accurate calculation, thereby selecting miRNAs with high sensitivity and high specificity different from those disclosed in the prior art, so that the miRNAs found by comprehensively analyzing the penalty index analysis of each miRNAs can best reflect the actual situation in clinical patients.

The combination, method and kit provided by the present invention can be used for screening and differential diagnosis of early pancreatic cancer, monitoring of disease complications and recurrence, evaluation of curative effect, drug efficacy and guidance of precise drug use, etc., and it has the advantages of a wide detection spectrum, high sensitivity, good specificity, low detection cost, convenient material acquisition, and easy storage of samples. This method can be widely used in the early screening and prognosis of pancreatic cancer, improving the low specificity and low sensitivity caused by the individual differences that are difficult to overcome due to the instability of a single marker or biomarkers currently widely used in clinical practice. It can significantly improve the clinical detection rate of early pancreatic cancer, reduce the misdiagnosis rate and missed diagnosis rate of pancreatic cancer, and become an effective method for early pancreatic cancer diagnosis.

### DESCRIPTION OF DRAWINGS

FIG. 1 Optimization flow of TRIzol LS extraction process.

FIGS. 2A and 2B qPCR reagent detection results, wherein FIG. 2A is the Vazyme reagent test result, FIG. 2B is the TAKARA qPCR reagent test result.

FIG. 3 is the human serum miRNA copy number change curve detected by PCR technology.

FIG. 4 shows the serum miRNA copy number change screened from normal person, pancreatitis and early pancreatic cancer patients screened by Combination 1 (*p<0.001).

FIG. 5 is the copy number change of Combination 3 and Combination 4 genes in human serum detected by PCR.

FIG. 6 is the decision tree analysis of the clinical trial results of Combination 1 (n=800).

FIG. 7 shows the Random Forest Model analysis of the clinical trial results of Combination 1 (n=800).

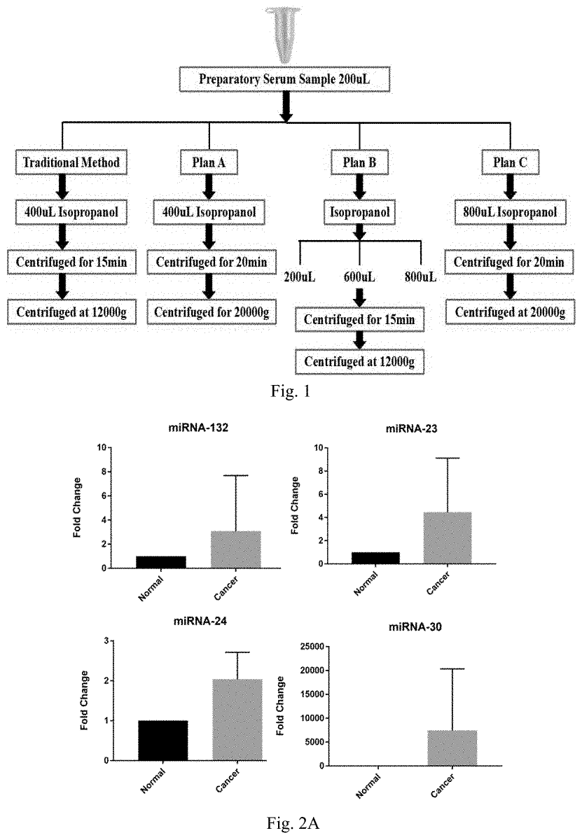

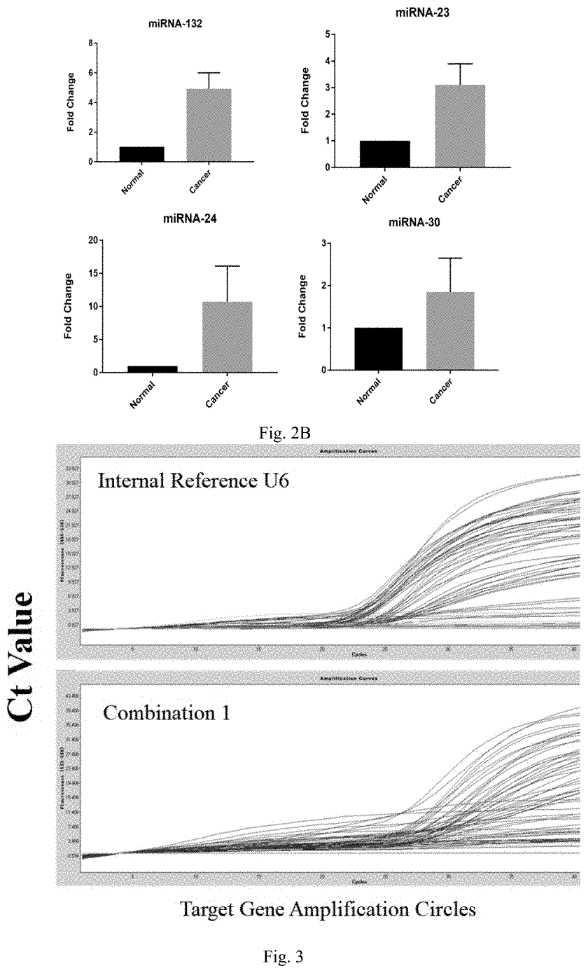

### Embodiment

The present invention is described in detail below through the accompanying drawings and specific embodiments, but the protection scope of the present invention is not limited. Unless otherwise specified, the experimental methods used in the present invention are all conventional methods, and the used experimental equipment, materials, reagents, etc. can be purchased from chemical companies.

### EXAMPLE 1

### Construction of Early Screening of miRNAs and the Precision Drug Detection System (SOP)

S1. Screen pancreatic cancer miRNAs, design specific probes and primers of the target miRNAs according to the principle of TaqMan probe technology; the target miRNAs is reacted with U6 Inter Reference in the same system; the designed primers and probes are as follows:

TABLE 1
Design the primer and probe
sequences of target miRNAs

| | | | |
|---|---|---|---|
| SEQ ID | miR-30c | Probe | TCAGCCCTATCACCG |
| NO. 1 | | | TTGTC |
| | | Forward | CGGTTGTAAACATCC |
| | | | TACACT |
| | | Reverse | GGGTCCGAGGTATCC |
| | | | AT |
| | | RT | CCTATCACCGTAAGC |
| | | primer | AGGGTCCGAGGTATC |
| | | | CATCGCACGCGTGGA |
| | | | CAACGGTGATAGGAT |
| | | | CTGC |
| SEQ ID | miR-24 | Probe | TCAACAGCTCCTATG |
| NO. 2 | | | GACGACC |
| | | Forward | CTTGGCTCAGTTCAG |
| | | | CAG |
| | | Reverse | GAGGTATCCATCGCA |
| | | | CG |
| | | RT | CTCCTATGGACAAGC |
| | | primer | AGGGTCCGAGGTATC |
| | | | CATCGCACGCGAGGG |
| | | | TCGTCCATAGGAGCT |
| | | | GTTCCT |
| SEQE ID | miR-23a | Probe | TCCCCAGCATAACCC |
| NO. 3 | | | TCG |
| | | Forward | TATCACATTGCCAGG |
| | | | GAT |
| | | Reverse | GGGTCCGAGGTATCC |
| | | | AT |
| | | RT | CCAGCATAACCAAGC |
| | | primer | AGGGTCCGAGGTATC |
| | | | CATCGCACGCATCCG |
| | | | AGGGTTATGCTGGGG |
| | | | AAAT |

S2. Optimization of miRNA extraction technology

(1) Determine the extraction reagent. Three commonly used commercial RNA isolation kits were screened, namely TRIzol (Ambion), TRIzol LS Reagent (Invitrogen) and miRNeasy Serum/Plasma kit (Qiagen). By comparing the quality of the extracted miRNA, and evaluating the cost and ease of operation degree and other factors, select the extraction reagent with the best comprehensive performance. The three extraction reagents were used to extract the same test sample (using Sw1990 pancreatic cancer cell line as an example) according to their instructions, and the extraction results were analyzed. As we can see from attached table 2, the RNA concentration extracted by TRIzol LS is the largest, and the RNA quality is more suitable, so we decided to use TRIzol LS Reagent (Invitrogen) as the extraction reagent of the present invention.

TABLE 2
Spectrophotometry and fluorescence quantitative PCR
analysis results of three miRNA extraction reagents

| Extraction | Concentration | | |
|---|---|---|---|
| reagent | (ng/μL) | OD260/280 | Ct Value |
| TRIzol | 23.840 ± 3.503 | 1.257 ± 0.033 | 21.937 ± 0.092 |
| TRIzol LS | 56.124 ± 3.900 | 1.780 ± 0.056 | 20.367 ± 0.198 |
| miRNeasy | 38.835 ± 0.756 | 1.840 ± 0.039 | 22.633 ± 0.007 |
| Serum/Plasma | | | |

(2) Optimizing the influencing factors in the extraction process. The extraction process of the kit that has been determined in the first step is improved and optimized to improve the quality of the isolated miRNA. The factors known to affect the extraction quality mainly include: the amount of lysate, the amount of chloroform, the amount of isopropanol, and the centrifugation conditions (time and rotation speed). The present invention mainly considers the influence of the amount of isopropanol and the centrifugal conditions on the extraction quality. An optimized flow chart of the TRIzol LS extraction process as shown in FIG. 1 was designed.

(3) The routine extraction method of TRIzol LS was optimized in detail according to the above three protocol, and 200 μL of serum samples from the same normal person were extracted. The extraction quality results are shown in Table 3 below. Through statistical analysis of the experimental results in Table 3, it can be seen that in Protocol A, compared with the traditional method, the concentration of extracted RNA increased by about 7-10 ng/μL. This shows that optimizing the centrifugation conditions, including increasing the speed and prolonging the centrifugation time, can fully precipitate the isolated RNA, increase the extraction concentration, and slightly improve the purity, but it is still outside of the optimal range (OD260/280=1.8˜2.0); Protocol B is to change the amount of isopropanol to 200 μL, 600 μL, and 800 μL under the same other conditions, and compare the quality of RNA extracted under these three gradient experimental conditions. It can be seen from the test results, the extraction effect was the best when the amount of isopropanol was 800 μL. Compared with the traditional method, the concentration was increased by 50.956±3.97ng/μL, not only that, the purity was significantly improved, its OD260/280 is about 1.9, which is in the optimum range for purity. Isopropanol precipitates RNA into pellets. This result shows that appropriately increasing the amount of isopropanol can significantly improve the extraction purity; Protocol C is a comprehensive use of the favorable factors of Protocol A and Protocol B, that is, changing the centrifugation conditions to 20000 g, 20 minutes and increasing the amount of isopropanol to 800 μL, the concentration and purity of the test results were significantly improved, which met the optimization purpose.

TABLE 3
Spectrophotometry and fluorescence quantitative PCR analysis
results of four protocols for extracting miRNA in serum

| | Concentration | | |
|---|---|---|---|
| Protocol | (ng/μL) | OD260/280 | Ct Value |
| Traditional Method | 94.356 ± 9.160 | 1.330 ± 0.120 | 35.313 ± 0.140 |
| Plan A | 101.585 ± 12.610 | 1.504 ± 0.011 | 34.993 ± 0.115 |
| | 105.971 ± 1.514 | 1.385 ± 0.006 | 34.837 ± 0.225 |
| Plan B | 106.955 ± 12.792 | 1.285 ± 0.139 | 34.593 ± 0.561 |
| | 145.312 ± 13.130 | 1.917 ± 0.028 | 32.817 ± 1.162 |
| Plan C | 167.431 ± 6.648 | 1.925 ± 0.131 | 33.947 ± 0.167 |

S3. Optimization and establishment of RT-PCR reaction procedure and reaction system. The multiplex RT-qPCR system established in the present invention is mainly divided into two parts of reaction, reverse transcription reaction (RT) and amplification reaction (PCR). Therefore, we adjusted the amount of RNA loaded in reverse transcription and the use of reagents in PCR amplification reactions, respectively.

(1) Optimization of RNA loading amount for reverse transcription: We set 5 gradient values for RNA loading amount: 50 ng(A), 25 ng(B), 12.5 ng(C), 6.25 ng(D), 3.125 ng(E), and then follow the instructions of the TaqMan™ MicroRNA Reverse Transcription Kit (ABI 4366596). Statistical analysis was performed on the experimental results of the five experimental groups of the four target miRNAs in Table 4, it can be seen that the four probes show a trend of increasing the Ct value significantly with the decrease of the RNA loading amount. Therefore, it is finally determined that the optimal loading amount of reverse transcribed RNA is 50 ng.

TABLE 4
Multiplex RT-qPCR results of four target miRNAs
with different RNA loading amounts in 5 groups

| | RNA Loading Amount | | | | |
|---|---|---|---|---|---|
| Target probe | 50 ng | 25 ng | 12.5 ng | 6.25 ng | 3.125 ng |
| miR-24-1-5p* | 30.35 | 30.74 | 30.36 | 32.69 | 34.75 |
| | 30.78 | 30.22 | 30.46 | 32.88 | 35.22 |
| miR-30c-5p | 30.17 | 32.3 | 34.42 | 35.97 | 37.05 |
| | 30.51 | 32.38 | 34.02 | 35.74 | 36.83 |
| miR-23a-3p | 29.21 | 31.29 | 34.57 | 33.82 | 37.34 |
| | 29.55 | 31.32 | 34.41 | 33.98 | 37 |
| miR-132-3p | 15.18 | 16.04 | 15.45 | 17.37 | 19.11 |
| | 16.29 | 15.37 | 15.95 | 17.89 | 18.86 |

Optimization of PCR amplification reaction reagents: we investigated two commonly used Taqman qPCR reagents on the market, and used the same samples (normal human serum and pancreatic cancer patient serum) for experimental comparison. The two reagents are AceQ qPCR Probe Master Mix (Vazyme) and Premix Ex Taq™ (Probe qPCR) (TAKARA), respectively, and operate according to the reagent instructions. Statistical analysis of the detection results of the two reagents shows that both reagents allow miRNAs to perform PCR reaction normally and distinguish between normal person and pancreatic cancer patient serum (compared with normal samples, the four miRNAs in pancreatic cancer patient serum with significantly high expression). However, the stability of Vazyme reagents in samples is not very good, often there will be one outlier in three replicate experiments, and it deviates far from the other two values (the error bar of the statistical graph is large). Therefore, the present invention adopts Premix Ex Taq™ (Probe qPCR) (TAKARA) as a qPCR reagent for clinical sample detection.

### Example 2

Using the multiple fluorescent probe detection technology of miRNA in serum established by the present invention, in the following examples, we detect miR-30c, miR-24, miR-23a, miR-132, miR-21, let The expressions of -7i, miR-1207, miR-130, miR-200c, miR-154, and miR-57 expression in a total of nearly 900 serum samples, and they were divided into 6 combinations, namely,

Combination 1: miR-24/miR-23a/miR-132/miR-30c;

Combination 2: miR-130/miR-200c/miR-154/miR-30c;

Combination 3: miR-24/miR-132/miR-1207/let-7i;

Combination 4: miR-30c/miR-24/miR-59/miR-132;

Combination 5: miR-130/miR-21/let-7i/miR-30c;

Combination 6: miR-30c/miR-154/miR-23a/miR-57.

Specifically: serum samples of patients from Shanghai Renji Hospital, Dalian Medical University, Peking Union Medical College Hospital and Nanjing Medical University (including early pancreatic ductal adenocarcinoma, pancreatitis, intraductal papillary mucinous tumor of pancreas) and serum samples of normal person from Hunan Xiangya Hospital, were operated according to the previously established SOP. The specific operation steps are:

### 1. Collection and Processing of Samples

(1). Required clinical samples (800 cases with detailed clinical follow-up data; sample size is based on statistical strength greater than 95%):

a) Cancer: early pancreatic cancer (<I stage) (200 cases), intermediate and advanced stage (>=II) (300)

b) Interference group:

\textcircled{1} Intraductal papillary mucinous neoplasm (IPMN) (50 cases);

\textcircled{2} Inflammation: (a) acute (30 cases), (b) chronic pancreatitis (50 cases)

\textcircled{3} Solid pseudopapillary tumor of pancreas (35 cases);

\textcircled{4} Pancreatic cystic adenoma (35 cases).

c) Normal: no cancer, no infectious diseases, no other metabolic diseases (100 cases).

(2). Sample type: serum 500 microliters/case
(3). Required clinical information:

\textcircled{1} Physiological information (sex, age, height, weight, smoking history, drinking history, family history of cancer, history of diabetes);

\textcircled{2} Pathological information (tumor location, tumor size, stage, histological grade, number of positive lymph nodes, and presence or absence of cancer metastasis);

\textcircled{3} Reference indicators (CA19-9, CA125, CEA, CA242);

\textcircled{4} Treatment plan (whether chemotherapy, chemotherapy plan, radiotherapy or not);

\textcircled{5} Follow-up information (follow-up time, survival status, recurrence or not, recurrence time, death time)

(4). Entry conditions: Only those who meet the following conditions can be enrolled:

\textcircled{1} Conform to the sample type in (1);

\textcircled{2} Have the clinical information required in (3);

\textcircled{3}The samples are well preserved and frozen in time without repeated freezing and thawing.

### 2. Preparation Before Experiment

Environment: The whole experimental process is operated in a clean room, and the normal temperature is 20-25 Celsius;
Instruments: high-speed centrifuge, Nanodrop, PCR amplification instrument, quantitative PCR instrument;
Consumables: RNAase-free 1.5 ml EP tube, 0.1 ml 8-strip PCR tube, 1 ml/200 ul/10 ul tips, 384-well plate;
Reagents: TRIzol™ LS Reagent (Invitrogen10296028), RNase-free ddH2O, TaqMan™ MicroRNA Reverse Transcription Kit (ABI 4366596), Premix Ex Taq™ (Probe qPCR) TAKARA RR390; chloroform, anhydrous ethanol, RT primer (U6, miR-30c, miR-24, miR-23a, miR-132), qPCR primer, probe (U6-Fam, VIC:miR-30c, miR-24, miR-23a, miR-132).

### 3. RNA Extraction—TRIzol™ LS Reagent

(1) Add 600 ul of TRIzol™ LS to each 200 ul serum sample, pipette tip repeatedly evenly, and incubate at room temperature for 5 minutes to fully lyse;

(2) Add 0.16 ml chloroform to the lysate, cover with a lid, and incubate at room temperature for 2-3 min;

Centrifuge at 20000 g, 4° C. for 20 min, the sample is divided into three layers, and the upper aqueous phase is transferred to a new centrifuge tube (be careful not to suck the middle layer);

(3) Add 800 μl isopropanol, cover with the lid and incubate at room temperature for 10 minutes;

(4) Centrifuge at 12,000×g for 10 min at 4° C., the RNA forms a white precipitate at the bottom of the tube, and remove the supernatant;

(5) Add 0.8 ml of 75% ethanol to resuspend and wash the precipitate;

(6) Centrifuge at 7500×g for 5 min at 4° C., remove the supernatant, and be careful not to aspirate the RNA precipitate;

(7) Open the tube lid and dry in the air for 5-10 min;

(8) Add 22 ul RNAase-free ddH₂O to dissolve RNA;

(9) Determine the concentration and the quality of the extracted RNA with Nanodrop.

### 4. Reverse Transcription-TaqMan™ MicroRNA Reverse Transcription Kit (ABI 4366596)

Combine any one of miR comprising miR-30c, miR-24, miR-23a, and miR-132, with U6, according to the ratio in the table, and prepare the working solution firstly, and then add the corresponding reaction reagent according to the ratio in the table to ensure that the total volume is 15 microliters. Then, PCR amplification experiments were performed. Prepare the following system in a 0.1 ml 8-strip PCR tube by pipetting and mixing, and prepare multiple samples together before aliquoting:

| Reagent | Amount (μl) |
|---|---|
| RNA | X (50 ng) |
| 10x Buffer | 1.5 |
| dNTP mix | 0.15 |
| RT enzyme | 1 |
| RNase inhibitor | 0.19 |
| U6 RT primer (5 μM) | 1 |
| miRNA RT primer (5 μM) | 1 |
| RNase-free ddH2O | 10.16-X |
| Total | 15 |

Perform the following procedure on the PCR amplifier: 16° C. 30 min→42° C. 30 min→85° C. 5 min→4° C., and centrifuge slightly to the bottom of the tube after completion.

### 5. RT-PCR-Premix Ex Taq™ (Probe qPCR) TAKARA RR390

Prepare the following system in a 0.2 ml PCR tube or RNAase-free 1.5 ml EP tube, pipetting and mixing, and aliquot into a 0.1 ml 8-strip PCR tube or a 384-well plate. cDNA is added to the tube wall separately, and when capping the 0.1 ml 8-strip PCR tube lid, do not touch the lid directly with your hands, and press down the lid with a piece of paper. If a 384-well plate is used, the sealing film is attached and centrifuged slightly to the bottom of the tube.

| Reagent | Amount (μl) |
|---|---|
| cDNA | 3 |
| Premix Ex Taq (Probe qPCR) (2×) | 5 |
| U6 Forward Primer (10 μM) | 0.2 |
| U6 Reverse Primer (10 μM) | 0.2 |
| miRNA Forward Primer (10 μM) | 0.2 |
| miRNA Reverse Primer (10 μM) | 0.2 |
| U6 Probe (10 μM) | 0.4 |
| miRNA Probe (10 μM) | 0.4 |
| RNase-free ddH2O | 0.4 |
| Total | 10 |

Premix Ex Taq can be stored at −20° C. for a long time, once thawed, please store at 4° C. and use up within 6 months.

PCR amplification was performed in a quantitative PCR instrument (Roche LC480II) using a two-step method. The reaction conditions were: pre-denaturation, 1 cycle, 95° C. for 30 seconds, PCR reaction, 40 cycles, 95° C. for 5 seconds and 60° C. for 30 seconds, anneal at 50° C. for 30 seconds, 1 cycle.

### QuantStudio DX Real-Time Fluorescent Quantitative PCR System

The reaction conditions were: pre-denaturation, 1 cycle, 95° C. for 30 seconds, PCR reaction, 45 cycles, 95° C. for 5 seconds and 60° C. for 40 seconds.

| Reagent | Amount (μl) |
|---|---|
| cDNA | 3 |
| Premix Ex Taq (Probe qPCR) (2×) | 5 |
| ROX Reference Dye (50×) | 0.2 |
| U6 Forward Primer (10 μM) | 0.2 |
| U6 Reverse Primer (10 μM) | 0.2 |
| miRNA Forward Primer (10 μM) | 0.2 |
| miRNA Reverse Primer (10 μM) | 0.2 |
| U6 Probe (10 μM) | 0.4 |
| miRNA Probe (10 μM) | 0.4 |
| RNase-free ddH2O | 0.2 |
| Total | 10 |

### 6. Pathological Status Analysis

Based on the quantitative PCR results performed with the core diagnostic combination, the distribution of biomarkers in the patient's blood samples was analyzed to determine the pathological status of the patient.

FIG. 3, taking the combination 1 as an example to illustrate the detection of the changes of the miRNA copy number in human serum (from normal person, pancreatitis and early pancreatic cancer patients) by PCR technology, from the results shown in the attached Figure, it can be seen that the internal reference positive control U6 is amplified signal in human serum samples, and the actual PCR signal for combination 1 to be tested and negative control miRNAs.

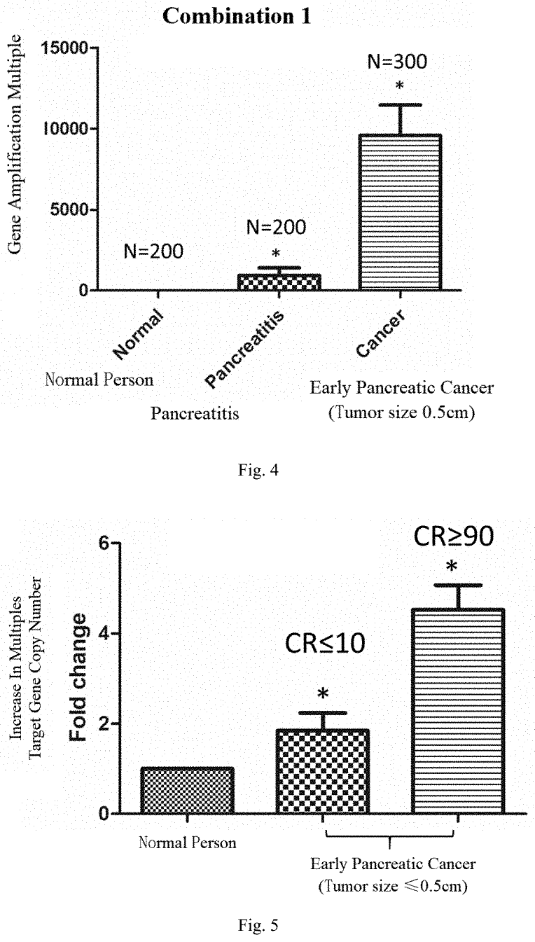

FIG. 4 shows the changes in serum miRNA copy number from normal person, pancreatitis and early pancreatic cancer patients screened by combination 1 (*p<0.001). The results shown in FIG. 4 show that the four miRNA markers in combination 1 can significantly differentiate early pancreatic ductal carcinoma, pancreatitis and normal person, so this combination is the best combination.

FIG. 5 shows the average gene copy number of either Combination 3 or Combination 4 genes, detected by PCR, in human serum of early pancreatic cancer patients (tumor size ≤0.5 CM) who were characterized as either sensitive (i.e., complete response (CR), CR [symbol] 90) or insensitive (i.e., CR [symbol] 10), to Gemcitabine treatment, PCR analysis of patient serum was performed before surgery, *p<0.001. From the results shown in the Figure, it can be seen that the expression levels (expressed as gene copy number, CN) of either Combination 3 or Combination 4 in the serum of pancreatic cancer patients can significantly distinguish the effectiveness of Gemcitabine therapy in different patients, that is, in patients who are effective on Gemcitabine therapy, the levels (e.g., CN) of either Combination 3 or 4 in their blood is greater at least 4 times higher than that in normal person blood (±0.25); or in patients who are ineffective to Gemcitabine treatment, the levels (e.g., CN) of either Combination 3 or 4 in their blood is at about twice higher than that in normal person blood.

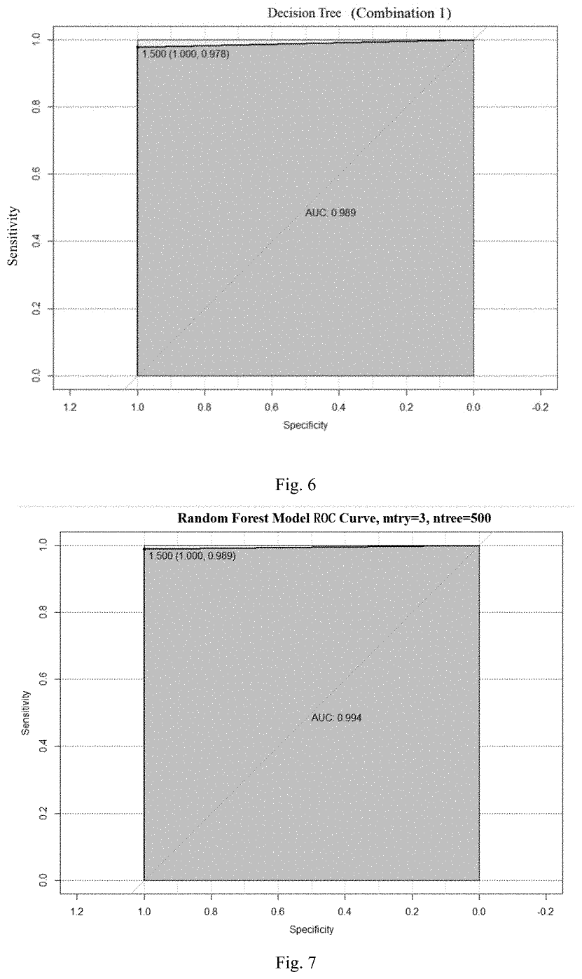

Quantitative PCR analysis was performed on miRNAs in the combined 1, together with clinical information of patients, and the distribution of biomarkers in the blood samples of patients was modeled and analyzed by big data informatics cloud computing method, so as to determine the pathological status of patients. The analysis of a clinical trial from 800 subjects is shown in FIG. 6-7. The results in FIG. 6 show that, using big data bioinformatics decision tree analysis, miRNAs in the combination 1 can significantly distinguish early pancreatic cancer from either pancreatitis, or intraductal papillary mucinous tumor or normal person. The sensitivity, specificity and accuracy (AUC) of the detection at early stages of pancreatic cancer as compared to normal healthy person was 100%, 97.8%, 98.9%, respectively.

In order to further verify the stability of the established model, the above test data combined with clinical information are verified by the random forest model (see FIG. 7). The results showed that Combination 1 can indeed significantly differentiate early pancreatic cancer, pancreatitis, intraductal papillary mucinous neoplasm and normal person. Its detection sensitivity was 100%, specificity was 98.9%, and accuracy (AUC) was 99.4%.

It can be seen from the above experimental data that the miRNAs combination of the present invention can distinguish early pancreatic cancer and benign intraductal papillary mucinous neoplasm, early pancreatic cancer and pancreatitis, benign intraductal papillary mucinous neoplasm and pancreatitis, and pancreatitis and normal tissue; and can accurately predict multidrug resistance (accuracy rate is about 90%), and the average clinical detection sensitivity is >98%.

The above is only a preferred embodiment of the present invention, but the protection scope of the present invention is not limited to this. The equivalent replacement or modification of the created technical solution and its inventive concept shall be included within the protection scope of the present invention.

What is claimed is:

1. A molecular marker for early pancreatic neoplasm detection, wherein miRNA of the pancreatic neoplasm molecular marker comprises but are not limited to: miR-30c, miR-24, miR-23a and miR-132.

2. The molecular marker for early pancreatic neoplasm detection according to claim 1, wherein miR-30c comprises hsa-miR-30c-5p; miR-24 comprises hsa-miR-24-3p; miR-23a comprises hsa-miR-23a-3p; miR-132 comprises hsa-miR-132-3p.

3. A molecular marker combination for early pancreatic neoplasm detection, comprising a core detection combination of any one or a combination of 2 to 4 of the miRNA according to claim 1.

4. The molecular marker combination for early pancreatic neoplasm detection according to claim 3, comprising:
Combination 1: miR-24/miR-23a/miR-132/miR-30c;
Combination 2: miR-130/miR-200c/miR-154/miR-30c;
Combination 3: miR-24/miR-132/miR-1207/let-7i;
Combination 4: miR-30c/miR-24/miR-59/miR-132;
Combination 5: miR-130/miR-21/let-7i/miR-30c;
Combination 6: miR-30c/miR-154/miR-23a/miR-57.

5. Use of the molecular marker according to claim 1, wherein the early pancreatic cancer detection marker is in a test kit or in any convenient detection method, comprises but not limited to a portable detection test paper, a digital detection strip/card and a detector, as well as a use of any chemical method to modify derivatives of the miRNA molecule.

6. The use according to claim 5, wherein the early pancreatic cancer detection marker for the kit or any convenient detection method, comprises its miRNA probe combination.

7. A method of early screening, precise drug detection system construction, based on the pancreatic neoplasm molecular marker according to claim 1, comprising steps:
S1. screening pancreatic cancer miRNA, designing a specific probe and a primer for the target miRNA according to a principle of TaqMan probe technology; using U6 or hsa-miR-16 or hsa-miR-159a as an internal reference to react with the target miRNA in a same system;
S2. optimization of miRNA extraction technology;
S2.1 determining an extraction reagent, according to a quality of miRNA extracted from a test sample by a separation kit, to select a best extraction reagent as TRIzol LS Reagent;
S2.2 optimizing an influencing factor in an extraction process, using a TRIzol LS Reagent kit to investigate effects of different isopropanol dosages and centrifugation conditions on the quality of extracted miRNA, and designing three detailed optimization schemes;
S2.3 optimizing details of a conventional extraction method of TRIzol LS according to the above scheme;
S3. optimization and establishment of multiplex RT-qPCR system reaction program and reaction system;
S3.1 RNA loading optimization for a reverse transcription;
S3.2 optimization of PCR Amplification Reagents;
using normal human serum and pancreatic cancer patient serum to proceed AceQ qPCR Probe Master Mix and Premix Ex Taq™ comparison experiments respectively, and selecting Premix Ex Taq™ as a qPCR reagent for a clinical sample detection.

8. The method according to claim 7, wherein in S3.1, a RNA optimum loading amount of the reverse transcription is 50 ng.

9. A clinical diagnosis method using the pancreatic neoplasm molecular marker according to claim 1, comprising steps:
S1. collecting a clinical sample and enrolling eligible cases;
S2. RNA extraction;
S3. RT-PCR reaction procedure and reaction system;
(1) combining miR comprising any one of miR-30c, miR-24, miR-23a, and miR-132, with U6, to prepare a reaction system in proportion, and then performing PCR amplification experiment; performing following procedures on a PCR amplification instrument: 16° C. 30 min→42° C. 30 min→85° C. 5 min→4° C., centrifuging slightly to the bottom of the tube after completion;
the reaction system is:

| Reagent | Amount (μl) |
|---|---|
| RNA | X (50 ng) |
| 10x Buffer | 1.5 |
| dNTP mix | 0.15 |
| RT enzyme | 1 |
| RNase inhibitor | 0.19 |
| U6 RT primer (5 μM) | 1 |
| miRNA RT primer (5 μM) | 1 |
| RNase-free ddH₂O | 10.16-X |
| Total volume | 15 |

(2) preparing a following system in a 0.2 ml PCR tube or RNAase-free 1.5 ml EP tube and mixing by pipetting

| Reagent | Amount (μl) |
|---|---|
| cDNA | 3 |
| Premix Ex Taq (Probe qPCR) (2×) | 5 |
| U6 Forward Primer (10 μM) | 0.2 |
| U6 Reverse Primer (10 μM) | 0.2 |
| miRNA Forward Primer (10 μM) | 0.2 |
| miRNA Reverse Primer (10 μM) | 0.2 |
| U6 Probe (10 μM) | 0.4 |
| miRNA Probe (10 μM) | 0.4 |
| RNase-free ddH₂O | 0.4 |
| Total volume | 10 |

using a two-step method to carry out PCR amplification, reaction conditions are: pre-denaturation, 1 cycle, 95° C. for 30 seconds, PCR reaction, 40 cycles, 95° C. for 5 seconds, 60° C. for 30 seconds, annealing at 50° C. for 30 seconds, 1 cycle;
(3) QuantStudio DX real-time quantitative PCR system, the reaction conditions are: pre-denaturation, 1 cycle, 95° C. for 30 seconds, PCR reaction, 45 cycles, 95° C. for 5 seconds and 60° C. for 40 seconds;

| Reagent | Amount (μl) |
|---|---|
| cDNA | 3 |
| Premix Ex Taq (Probe qPCR) (2×) | 5 |
| ROX Reference Dye (50×) | 0.2 |
| U6 Forward Primer (10 μM) | 0.2 |
| U6 Reverse Primer (10 μM) | 0.2 |
| miRNA Forward Primer (10 μM) | 0.2 |
| miRNA Reverse Primer (10 μM) | 0.2 |
| U6 Probe (10 μM) | 0.4 |
| miRNA Probe (10 μM) | 0.4 |
| RNase-free ddH₂O | 0.2 |
| Total volume | 10 |

S4. according to the quantitative PCR results performed with a core diagnostic combination, analyzing a distribution of biomarkers in a blood sample of a patient, and determining a pathological status of the patient.

10. The method according to claim 9, wherein step S2 is specifically:
(1) adding 600 ul TRIzol™ LS to each 200 ul serum sample and incubating at a room temperature to fully lyse;
(2) adding chloroform to the lysate, and incubating at the room temperature;
centrifuging at 20,000 g, 4° C. for 20 min, and transferring an upper aqueous phase to a new centrifuge tube;
(3) adding 800 μL of isopropanol, and incubating at the room temperature; centrifuging at 12,000×g for 10 min at 4° C., RNA forms a white precipitate at a bottom of the tube, removing a supernatant; adding 75% ethanol to resuspend and washing the precipitate; 7500×g, centrifuging at 4° C. for 5 min, removing the supernatant and air dry; adding ddH₂O to dissolve RNA; determining a concentration and a quality of the extracted RNA.


SEQUENCE LISTING (16 sequences)

SEQ ID NO: 1 - RNA, 20 nt, Artificial Sequence
  Synthesized
ucagcccuaucaccguuguc

SEQ ID NO: 2 - RNA, 21 nt, Artificial Sequence
  Synthesized
cgguuguaaacauccuacacu

SEQ ID NO: 3 - RNA, 17 nt, Artificial Sequence
  Synthesized
ggguccgagguauccau

SEQ ID NO: 4 - RNA, 64 nt, Artificial Sequence
  Synthesized
ccuaucaccguaagcaggguccgagguauccaucgcacgcguggacaacggugauaggaucugc

SEQ ID NO: 5 - RNA, 22 nt, Artificial Sequence
  Synthesized
ucaacagcuccuauggacgacc

SEQ ID NO: 6 - RNA, 18 nt, Artificial Sequence
  Synthesized
cuuggcucaguucagcag

SEQ ID NO: 7 - RNA, 17 nt, Artificial Sequence
  Synthesized
gagguauccaucgcacg

SEQ ID NO: 8 - RNA, 66 nt, Artificial Sequence
  Synthesized
cuccuauggacaagcaggguccgagguauccaucgcacgcgagggucguccauaggagcuguuccu

SEQ ID NO: 9 - RNA, 18 nt, Artificial Sequence
  Synthesized
uccccagcauaacccucg

SEQ ID NO: 10 - RNA, 18 nt, Artificial Sequence
  Synthesized
uaucacauugccagggau

SEQ ID NO: 11 - RNA, 17 nt, Artificial Sequence
  Synthesized
ggguccgagguauccau

SEQ ID NO: 12 - RNA, 64 nt, Artificial Sequence
  Synthesized
ccagcauaaccaagcaggguccgagguauccaucgcacgcauccgaggguuaugcuggggaaau

SEQ ID NO: 13 - RNA, 26 nt, Artificial Sequence
  Synthesized
uuccgccugcuugacuguagccgacg

SEQ ID NO: 14 - RNA, 21 nt, Artificial Sequence
  Synthesized
uuccuaacagucuacagccau

SEQ ID NO: 15 - RNA, 17 nt, Artificial Sequence
  Synthesized
ggguccgagguauccau

SEQ ID NO: 16 - RNA, 66 nt, Artificial Sequence
  Synthesized
ccugcuugacuaagcaggguccgagguauccaucgcacgucggcuacagucaagcaggcgaccaug